(19) United States
(12) Patent Application Publication
(10) Pub. No.: US 2022/0215920 A1
(43) Pub. Date: Jul. 7, 2022

(54) AUGMENTED INTELLIGENCE SYSTEM FOR SYMPTOM TRANSLATION

(71) Applicant: James P Tafur, South Jordan, UT (US)

(72) Inventor: James P Tafur, South Jordan, UT (US)

(21) Appl. No.: 17/563,020

(22) Filed: Dec. 27, 2021

Related U.S. Application Data
(60) Provisional application No. 63/133,329, filed on Jan. 2, 2021.

(51) Int. Cl. G16H 15/00; G16H 50/20; G16H 50/50; G16H 50/30

(52) U.S. Cl. CPC G16H 15/00; G16H 50/30; G16H 50/50; G16H 50/20

(57) ABSTRACT

Disclosed herein is a system and method for translating patient symptoms into one or more probable medical conditions. The method uses one or more individual knowledge data models in form of knowledge graphs to output a given medical condition based on input of patient symptoms. The individual knowledge data model is trained using the clinical decision-making logic of a healthcare professional and is continuously updated with inputs from a shared model and a world model. The knowledge data model outputs a medical condition analysis report that lists one or more probable medical conditions and a list of healthcare professionals specializing in treatment of said probable medical conditions. Further, aa curated list of educational and medical literature pertaining to each of the said probable medical conditions is provided for further research by the patient and the healthcare professional.

### FIELD OF INVENTION

Embodiments of a present disclosure relate generally to data processing systems and methods and specifically to a system and method for medical information processing and symptom translation using supervised learning methods.

### BACKGROUND

Medical science and research have reached greater heights in the recent times and the healthcare professionals are now equipped with greater medical knowledge to help overcome medical conditions of patients and offer guided treatment. However, one of the basic analysis that initiates the treatment course is identification of disease. Once the disease is diagnosed correctly, treatment becomes much easier. However, correct diagnosis is a tough task due to complex nature of symptoms and biological systems. If the diagnosis is not correct, a patient is at risk of not being treated correctly for the disease and hence the disease remains with the patient. Another risk of wrong diagnosis is that the patient might be mistreated resulting in unwanted medical side effects and medications. Both situations might be fatal to a patient.

Conventionally, medical conditions are identified by health care professionals based on the symptoms disclosed by a patient. The healthcare professional, based on the disclosed symptoms by the patient, determines the medical condition based on his clinical decision-making logic. This clinical decision-making logic is generally formulated by the healthcare professional through experience treating medical conditions and associated symptoms amongst various patients.

However, diagnosis by translating symptoms into probable diseases based on experience and subjective recollection is fret with potential shortcomings. The healthcare professional might not have encountered a particular symptom for a disease in his limited experience and that may lead to incorrect diagnosis. Further, the healthcare professional may in some cases mistakenly apply t wrong clinical logic due to hurry or some other human/clerical issues which again leads to misdiagnosis. Furthermore, in some cases, the healthcare professional might not be updated with latest diseases and corresponding symptoms which would lead to diagnosis being left incomplete.

Therefore, in light of the abovementioned shortcomings associated with conventional methods of diagnosis of diseases from symptoms, there is a need for a system and method for translation of symptoms into one or more probable medical conditions in an accurate, efficient and automated manner.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

The present invention discloses a system and method for translating patient symptoms into one or more probable medical conditions. The method uses one or more individual knowledge data models in form of knowledge graphs to output a given medical condition based on input of patient symptoms. The individual knowledge data model is trained using the clinical decision-making logic of a healthcare professional and is continuously updated with inputs from a shared model and a world model. The knowledge data model outputs a medical condition analysis report that lists one or more probable medical conditions and a list of healthcare professionals specializing in treatment of said probable medical conditions. Further, aa curated list of educational and medical literature pertaining to each of the said probable medical conditions is provided for further research by the patient and the healthcare professional.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary system capable translating symptoms into probable medical conditions, in accordance with an embodiment of the present disclosure.

FIG. 2 depicts a model mapping form as per the present disclosure.

FIG. 3 depicts a shared knowledge data model.

FIG. 4 depicts an exemplary a patient assessment form and recorded medical condition information; and

FIG. 5 depicts an example condition analysis report as per the present disclosure.

FIG. 6 depicts a flowchart for the method steps of the present invention.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting. A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

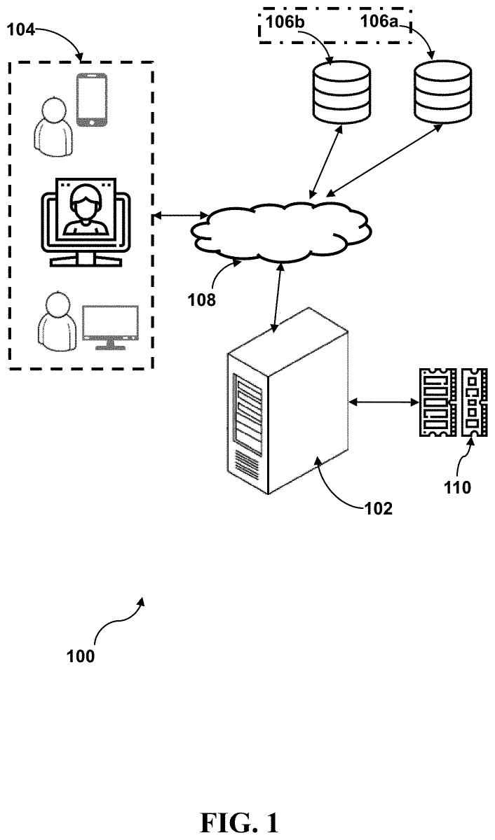

Referring now to the drawings, and more particularly to FIG. 1 through FIG. 6, where similar reference characters denote corresponding features consistently throughout the figures, there are shown preferred embodiments and these embodiments are described in the context of the following exemplary system and/or method.

FIG. 1 is a block diagram illustrating an exemplary system 100 capable translating symptoms into probable medical conditions, in accordance with an embodiment of the present disclosure. The invention discloses a system for patient symptom translation into biological systems and potential health conditions. The system 100 comprises a processing unit 102 communicably coupled to a client device 104 and a plurality of databanks (106a, 106b) through a data communication network 108. The processing unit 102 comprises a memory 110 that contains executable machine-readable instructions. The processing unit resides on a central server such as a cloud server or a remote server. In an exemplary embodiment of the present disclosure, the data communication network 108 may be internet. In the exemplary embodiment of the present disclosure, the client device 104 is associated with a graphical user interface and may include a laptop computer, desktop computer, tablet computer, smartphone, wearable device, smart watch and the like. Furthermore, the client device 104 include a web browser, a mobile application or a combination thereof to access the processing unit 102 via the data communication network 108. In an embodiment of the present disclosure, the client device 104 may use a web application through the web browser to access the processing unit 102. The mobile application may support android platforms, IOS platforms or both.

The client device 104 can be accessed by a healthcare professional or a patient to input medical condition information. Alternatively, the client device 104 can be used by a healthcare professional to input clinical decision-making logic.

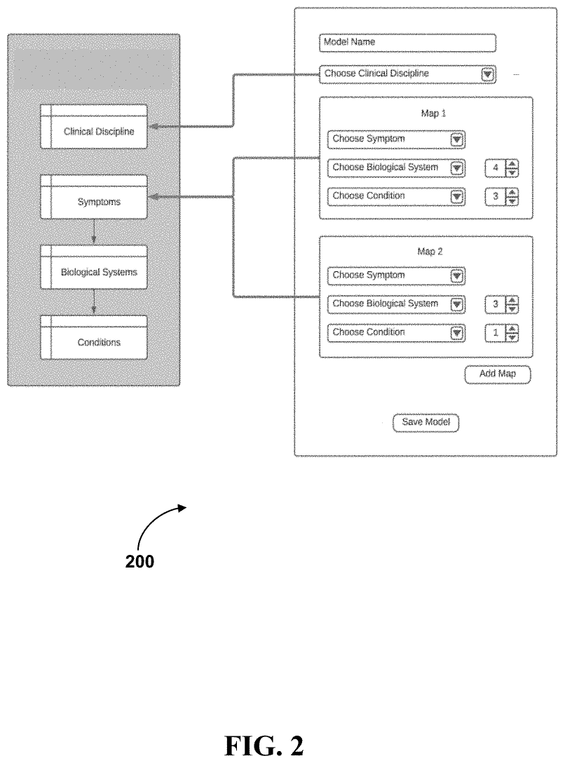

In an aspect of the present invention, a healthcare professional inputs his clinical decision-making logic through a model mapping form accessible though the client device 104 to create a knowledge data model. FIG. 2 depicts a model mapping form 202 as per the present disclosure. The healthcare professional, based on his clinical decision-making experience, inputs a list of symptoms and associated biological systems such as nutrition deficiency, mitochondria, immune response etc. and medical conditions such as Hunter disease, ALS Syndrome etc. The said information is saved against a clinical discipline and called a knowledge data model 204. The knowledge data model contains several such models based on experience of the healthcare professional. The knowledge data model 204 is searchable based on clinical discipline such as Psychology, Anaesthesia etc. The knowledge data model further comprises a measure of severity of symptoms recorded under a given medical condition. The knowledge data model is modelled as a knowledge graph capturing the relationship between medical condition, impacted biological systems, symptoms and associated severity of symptoms within a given clinical discipline. The knowledge graph is based on logical and semantic relationships between the symptoms, conditions and clinical discipline. In an embodiment, the knowledge data model is supervised learning based data model with labelled training data input in form of healthcare professional's clinical decision making logic. The knowledge data model is then operable to output a probable medical condition given a list of symptom and severity of the symptom. A mapping function is developed based on the clinical decision-making logic of the healthcare professional. The output, in form of probable medical condition, is derived by inputting symptoms and associated severity level available from the medical condition report. It shall be appreciated by persons skilled in the art that knowledge data models may be support vector machines (SVMs) and probabilistic classifiers (naïve Bayes) or any other suitable supervised learning algorithm and all such algorithms are covered within the scope of this disclosure.

The knowledge data model specific to each healthcare professional is termed as individual model as per the present disclosure.

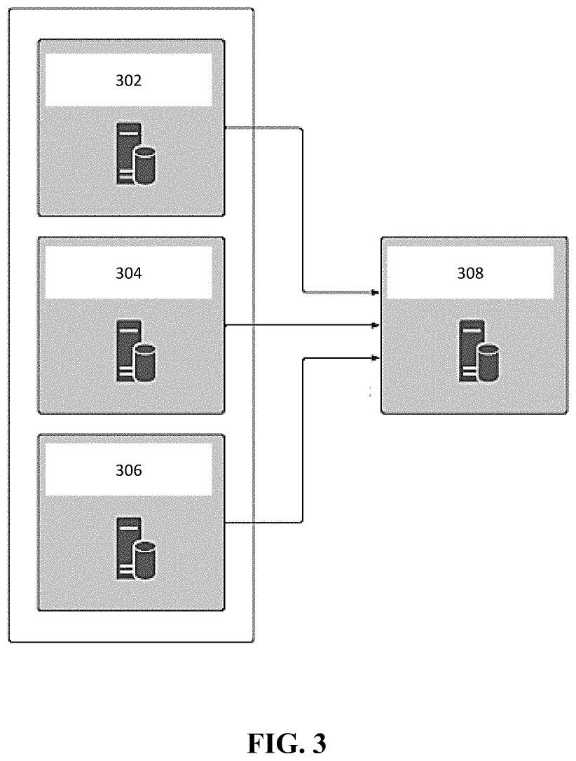

In yet another aspect of the present disclosure, knowledge data models of two or more healthcare professional can be collated to form a shared model. The shared knowledge data mode is created by calculated average of two or more individual data models. Shared models help remove biases in individual knowledge data models and healthcare professional can take help of experience of other healthcare professional's experience to refine and update their own knowledge data models. FIG. 3 depicts a shared knowledge data model. 302, 304 and 306 are individual models that are collated to forma shared model 308.

In another aspect of the present disclosure, a knowledge data model termed as world model is prepared based on training data provided by one or more individual models and patient treatment data outcome corresponding to the individual model. The world model is a continuously updating model based on inputs from various healthcare professional's individual models and patients inputting their outcomes. The world model can be depicted as combining all the information available from individual models and shared models into one model.

The clinical decision-making logic of individual model can be updated based on recent findings and treatment outcomes for patients. Further, individual models can be updated based on findings of shared models and results from world model. Updating individual model includes modifying symptoms, their severity level or associated medical conditions. Updating individual model results in enhanced efficiency of individual models due to inputs from peer's experience and enhanced knowledge base of world model. This change in training data for the supervised learning-based data model results in refinement of individual data models and leads to accurate identification of probable medical conditions.

The plurality of databanks 106a and 106b are repositories comprising a curated list of educational and medical literature pertaining to a given medical condition. In an embodiment, the databank stores the said educational and medical literature for instant retrieval. Optionally, the databank provides links to said curated list instead of storing actual documents. At least one of the plurality of databank comprises a list of healthcare professionals grouped by area of specialization wherein each area of specialization corresponds to a clinical discipline and medical condition. Each of the databank are searchable based on a medical condition. As an example, person X specializing as neurologist and treating conversion disorders and another person Y specializing as neurologist and treating neuro-spinal disorders. In another example, medical condition of Hunter disease returns a set of educational and medical literature pertaining to Hunter disease and a list of healthcare professionals specializing in treatment of hunter disease. The one or more databanks may be a cloud storage or a local file directory within a remote server.

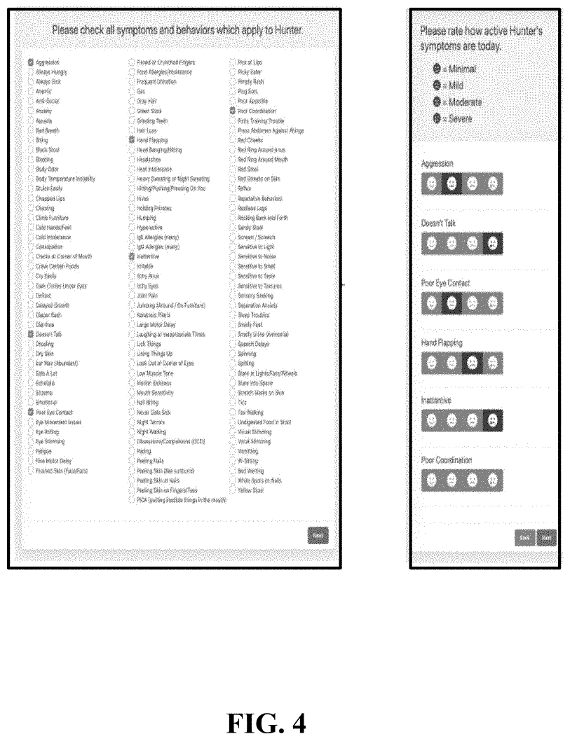

Referring to FIG. 1, the processing unit 102 is operable to receive, through the client device, medical condition information of a patient. The medical condition information pertains to one or more symptoms and associated severity level for each of the symptom. The medical condition information is entered in form of key-pair format such as symptom and a severity level for each symptom. As a non-limiting example, the severity level of symptoms can be one of minimal, mild, moderate and severe. The healthcare professional fills up a patient assessment form based on response received from the patient regarding the symptoms and the associated severity level. The patient assessment form comprises al list of medical conditions to choose from. Each of the medical conditions is associated with a list of symptoms clinically known to be associated with the medical condition. FIG. 4 depicts an exemplary a patient assessment form and recorded medical condition information.

In another embodiment of the present invention, the patient is enabled to input the medical condition information through a patient assessment form accessible through the client device in form of a remote patient monitoring system. The client device at patient's end is communicably coupled with the processing unit 102. The patient can remotely enter the medical condition information without the presence of healthcare professional. Optionally, the patient may record and update a treatment outcome through the remote patient monitoring system.

The processing unit 102 is further operable to perform a mapping of the received medical condition information against a knowledge data model to identify one or more probable medical conditions of the patient. In an aspect of the present invention, the knowledge data model is one of an individual model, a shared model or a world model. The knowledge data model is selected by the healthcare professional. The mapping comprises semantic similarity between the symptoms from the medical condition information and the corresponding symptoms in the knowledge data model. Based on the symptoms provided in the medical condition information, one or more probable medical conditions with similar symptoms and severity levels are identified. In some cases, the output is a single medical condition whereas in some cases, there could more than one medical conditions with similar symptoms and severity levels.

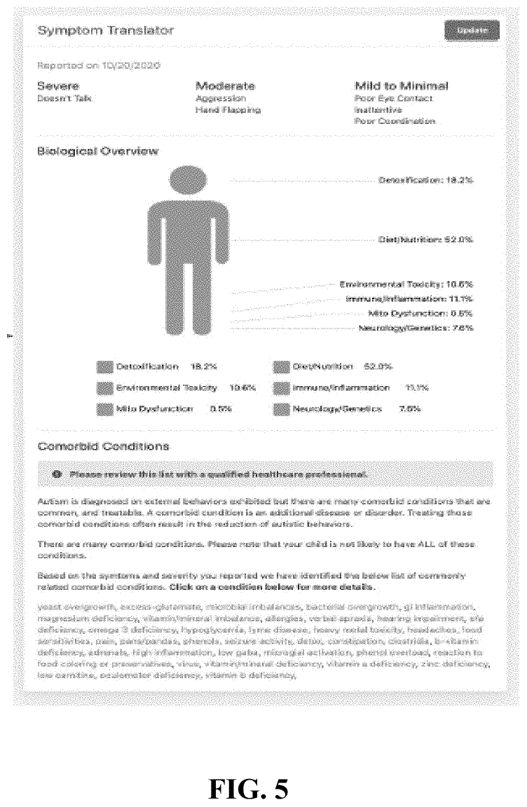

The processing unit 102 is further operable to prepare a condition analysis report for the received medical condition information. The condition analysis report comprises a list of probable medical conditions, a biological system impact chart, a set of curated educational material corresponding to the each of the probable medical condition and a list of healthcare professional specialized in treating each of the probable medical condition. FIG. 5 depicts an example condition analysis report as per the present disclosure. The probable medical conditions are retrieved from the mapping of symptoms of medical condition information against the knowledge data model. The probable medical conditions are sorted based on increasing similarity score as outputted by the knowledge data model. The set of curated educational material is retrieved from one of the plurality of databank based on the probable medical condition. Similarly, the list of healthcare professionals specialising in treatment of the said medical condition is retrieved from another databank.

The processing unit 102 is further operable to display the condition analysis report on a graphical user interface associated with the client device. The patient and the healthcare professional can view the condition analysis report through the graphical user interface and further decide on the treatment options. Optionally, the condition analysis report is displayed to the remote patient monitoring device associated with a client device a patient's end.

In yet another aspect of the present invention, the output of the knowledge data model is compared against a shared data model as well as the world data model to validate an accuracy of condition analysis report. In case of shared data model, two or more individual models are input with the received medical condition report and the output is compared with the condition analysis report from the individual model. Similarly, the world model is fed with the medical condition report and the output is compared with the condition analysis report. In case, the shred mode and the world model provide different output, the condition analysis report can be presumed to be inaccurate. The processing unit 102 allows a healthcare professional to update an individual mode based on the output of the shared model and the world model. The updated conditional analysis report is then prepared and displayed on the graphical user interface of the client device.

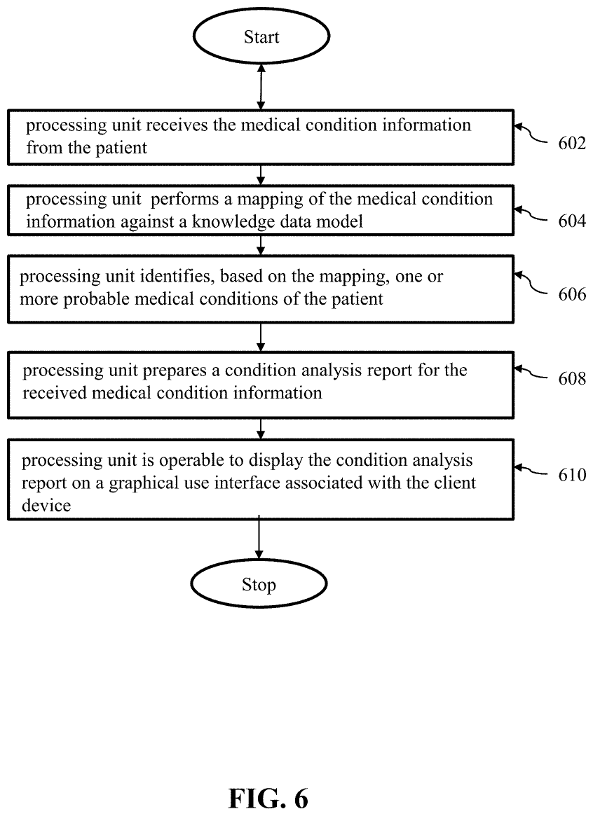

FIG. 6 depicts a flowchart 600 for the method steps of the present invention performed by the processing unit 102. A patient inputs, using a patient assessment chart available through a client device 104, medical condition information in form of one or more symptoms and associated severity of symptoms. At step 602, the processing unit 102 receives the medical condition information from the patient. At step 604, the processing unit 102 performs a mapping of the medical condition information against a knowledge data model. At step 606, the processing unit identifies, based on the mapping, one or more probable medical conditions of the patient. At step 608, the processing unit 102 prepares a condition analysis report for the received medical condition information. Finally, at step 610, the processing unit 102 is operable to display the condition analysis report on a graphical use interface associated with the client device 104.

The processing unit 102, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The processing unit 102 may also include embedded controllers, such as generic or programmable logic devices or arrays, application specific integrated circuits, single-chip computers, and the like.

The memory 110 may be non-transitory volatile memory and non-volatile memory. The memory 110 may be coupled for communication with the processing unit 102, such as being a computer-readable storage medium. The processing unit 102 may execute machine-readable instructions and/or source code stored in the memory 110. A variety of machine-readable instructions may be stored in and accessed from the memory 110. The memory 110 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 110 includes the plurality of modules stored in the form of machine-readable instructions on any of the above-mentioned storage media and may be in communication with and executed by the processing unit 102.

The data communication network 108 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of the foregoing.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items.

I/We claim:

1. A system for patient symptom translation into biological systems and potential health conditions, the system comprising a processing unit communicably coupled, through a data communication network, to a client device and one or more databanks, wherein the processing unit is configured to execute machine readable instructions that cause the processing unit to:
receive, through the client device, medical condition information of a patient wherein the medical condition information pertains to one or more symptoms along with the severity level;
perform a mapping of the medical condition information against a knowledge data model to identify one or more probable medical conditions of the patient;
prepare a condition analysis report for the received medical condition information; and
display the condition analysis report on a graphical user interface associated with the client device.

2. The system of claim 1 wherein the mapping comprises semantic similarity between the symptoms from the medical condition information and the corresponding symptoms in the knowledge data model.

3. The system of claim 1 wherein the knowledge data model is an individual model.

4. The system of claim 1 wherein the knowledge model is a shared model created by calculated averages of two or more individual data models.

5. The system of claim 1 wherein the knowledge data model is a world model prepared based on training data comprising one or more individual models and patient treatment outcome data corresponding to the individual model.

6. The system of claim 4 wherein the individual model is created by a healthcare professional inputting clinical decision-making logic using a model mapping form and wherein the clinical decision-making logic of individual model is refined based on patient treatment outcome data and comparison between a shared model and a world model.

7. The system of claim 1 wherein the condition analysis report comprises a list of probable medical conditions, a biological system impact chart, a set of curated educational material corresponding to the each of the probable medical condition and a list of healthcare professional specialized in treating each of the probable medical condition.

8. The system of claim 1 wherein the condition analysis report prepared using individual knowledge data model is compared to shared knowledge data model and world data model to validate an accuracy of the condition analysis report.

9. The system of claim 1 wherein the client device is a remote patient monitoring system and the medical condition information is received through a patient assessment form.

10. A method for patient symptom translation into biological systems and potential health conditions, the system comprising a processing unit communicably coupled, through a data communication network, to a client device and one or more databanks, wherein the processing unit is configured to execute machine readable instructions that cause the processing unit to:
receive, through the client device, medical condition information of a patient wherein the medical condition information pertains to one or more symptoms along with the severity level;
perform a mapping of the medical condition information against a knowledge data model to identify one or more probable medical conditions of the patient;
prepare a condition analysis report for the received medical condition information; and
display the condition analysis report on a graphical user interface associated with the client device.

11. The method of claim 10 wherein the mapping comprises semantic similarity between the symptoms from the medical condition information and the corresponding symptoms in the knowledge data model.

12. The method of claim 10 wherein the knowledge data model is an individual model.

13. The method of claim 10 wherein the knowledge model is a shared model created by calculated averages of two or more individual data models.

14. The method of claim 10 wherein the knowledge data model is a world model prepared based on training data comprising one or more individual models and patient treatment outcome data corresponding to the individual model.

15. The method of claim 12 wherein the individual model is created by a healthcare professional inputting clinical decision-making logic using a model mapping form and wherein the clinical decision-making logic of individual model is refined based on patient treatment outcome data and comparison between a shared model and a world model.

16. The method of claim 10 wherein the condition analysis report comprises a list of probable medical conditions, a biological system impact chart, a set of curated educational material corresponding to the each of the probable medical condition and a list of healthcare professional specialized in treating each of the probable medical condition.

17. The method of claim 10 wherein the condition analysis report prepared using individual knowledge data model is compared to shared knowledge data model and world data model to validate an accuracy of the condition analysis report.

18. The method of claim 10 wherein the client device is a remote patient monitoring system and the medical condition information is received through a patient assessment form.
